(19) United States
(12) Patent Application Publication
(10) Pub. No.: US 2012/0136081 A1
(43) Pub. Date: May 31, 2012

(54) Resorbable Polyurethane Wound Cover

(75) Inventors: Jessica Blume, Zurich (DE); Stephan Buser, Sursee (CH); Andreas Dobmann, Oberkirch (CH); Erika Zimmermann, Buochs (CH)

(73) Assignee: NOLAX AG, Sempach Station (CH)

(21) Appl. No.: 13/388,188

(22) PCT Filed: Aug. 3, 2010
(86) PCT No.: PCT/EP2010/061254

§ 371 Date: Jan. 31, 2012

(30) Foreign Application Priority Data
Aug. 3, 2009 (EP) 09167043.0

(51) Int. Cl. A61L 15/42; C08J 9/04

(52) U.S. Cl. 521/78; 521/88; 521/84.1; 264/54

(57) ABSTRACT

The present invention relates to a biocompatible, resorbable polyurethane foam wound cover with open pores and an adjustable resorption rate having improved properties when used on skin or connective tissue wounds as well as a method of production of such a polyurethane foam wound cover.

The present invention relates to a biocompatible, resorbable polyurethane foam wound cover with open pores and an adjustable resorption rate having improved properties when used on skin or connective tissue wounds as well as a method of production of such a polyurethane foam wound cover. The polyurethane foam wound cover can be individually tailored and placed on a wound or into a wound cavity to act as a scaffold for new cells. The resorption rate of the polyurethane foam wound cover can be adapted to the wound properties individually.

The use of polyurethane foams in medicine and especially as wound covers is known. When used as wound covers, foams can protect the wound from drying out, they can absorb wound exudate or they can serve as matrix for active substances or as scaffold for the ingrowth of new skin or connective tissue cells.

As wound cover is understood a material which can be used to cover a wound (topical application) as well as a material which is suitable to be inserted into a wound cavity.

Wound covers made of polyurethane foams are available in different forms. Some of them are self-adhesive, while others need an external fixation e.g. with an adhesive tape or with a gauze. Such foams are available as plates or rolls in different sizes. They can be adapted to different wound sizes e.g. through cutting (R. A. Ryant, D. P. Nix, Acute and chronic wounds: current management concepts, Elsevier Health Sciences, 2007, chapter 19, page 411 following).

For example U.S. Pat. No. 3,978,266, U.S. Pat. No. 3,975,567 and EP 0 059 048 describe polyurethane foams for topical application on wounds.

WO 88/01878 describes self-adhesive polyurethane foams which may comprise methacrylate incorporated by polymerisation. The foam may be applied onto another polymer foil to be used as wound cover. Further, the possibility to include active substances, such as antibiotics or growth factors in the foam is being mentioned.

EP 1 981 550 describes a method to apply a germ barrier made of a foil comprising a polymeric material and a wound cover made thereof. The method includes application of a thermoplastic material directly on a polymeric material to produce the wound cover.

A major disadvantage of these polyurethane foams is that they are not resorbable and have therefore to be completely removed from the body. This causes stripping of newly ingrown cells from the wound each time the wound cover is changed. Re-sorbable polyurethane foams as described herein can be used as scaffold for cells growing into a wound or a wound cavity in the sense of an extra cellular matrix thereby considerably increasing the rate of wound healing.

Polyurethane foam is resorbable by the body if the foam can be disintegrated trough hydrolysis, oxidation or enzymatic cleavage. Hydrolysis occurs when the foam is exposed to a water containing media, especially wound exudate. Oxidation reactions occur through the action of free oxygen radicals which may be released by macrophages during an inflammation reaction. Macrophages also secrete the enzyme cholesterol esterase which cleaves the ester bonds of the polyurethane foam. However the main resorption mechanism of resorbable biopolymers is believed to be hydrolysis (S. A. Guelcher, Biodegradable Polyurethanes: Synthesis and Applications in Regenerative Medicine, Tissue engineering: Part B, 2008, volume 14, no. 1; E. M. Christenson, S. Patel, J. M. Anderson, A. Hiltner, Enzymatic Degradation of Poly(ether urethane) and poly (carbonate urethane) by cholesterol esterase, Biomaterials, 2006, vol. 23, 3920-3926).

US 2007/0299151 describes a biocompatible and resorbable polyurethane foam. This application describes a polyurethane foam which is produced by reacting a biocompatible polyol, especially polycaprolactone, with at least one isocyanate, water, one or more stabilisers as well as a pore forming agent. This polyurethane foam composition is intended to be applied as a fluid on a bone fracture where it will harden into a polyurethane foam with open pores and a low resorption rate. A polyurethane foam to be used as extra cellular matrix for bone tissue requires other characteristics than a polyurethane foam to be used on skin or connective tissue wounds. Foams to be used as filler material for bone fractures need to have a compressive strength of between 0.05-100 MPa such as to match the rigidity and hardness of native bone tissue. The resorption rate also needs to be adapted to the slower cell growth rate in bone, since the regeneration of a fracture e.g. of the femur requires between 8 and 12 weeks compared to the 2 to 3 weeks needed for the regeneration of a skin or connective tissue wound.

A polyurethane foam to be used as an extra cellular matrix on skin or connective tissue wounds requires other characteristics. The foam has to be soft (compressive strength preferably less than 0.005 MPa when wet) and has to be tailorable to the wound bed. Moreover, the resorption rate should be adapted to the growth rate of skin or connective tissue cells. The resorption rate therefore needs to be in the range of between 2 days to 4 weeks.

According to the norm DIN EN ISO 7726 foams can be categorized according to their compressive strength and the characteristics of their pores. According to this norm, a pore is a small cavity which is completely or partially surrounded by pore walls and/or pore bridges. An open pore is a pore which is in fluid connection with other pores. The relative amount of open and closed pores can be determined according to the norm DIN EN ISO 4590.

Wounds can be categorized into acute and chronic wounds. Acute wounds are lesions and postoperative wounds which are healing at a normal rate. Wounds showing only a very slow healing rate or no healing at all after 6 to 8 weeks or categorized as chronic wounds. Such chronic wounds are mostly prevalent as secondary diseases with patients having diabetes mellitus or coronary diseases. The mostly occurring chronic wounds are ulcus cruris venosum, arterial ulcus, diabetic foot syndrome and decubitus. Chronic wounds show a large variation in their dimensions and depth, they can be infected and do also produce varying amounts of exudate. Especially in the case of decubitus deep wounds occur since tissue layers are detached from one another. These deep wound cavities can not be readily inspected by medical personal and it is therefore very difficult to decide on the appropriate treatment to be administered. A wound cover on a wound showing a heavy production of exudate needs to be changed frequently whereas a cover on a wound showing less exudate production might be changed only once a week.

WO 2006/032501 describes a polyurethane foam with open pores having no skin formation on its surface. This foam can be used as scaffolding for cell and tissue cultures or as a carrier for drugs. Formation of open pores is achieved by the use of a polysaccharide component present in an amount of between 0.01-4.2% by weight.

Implants and resorbable materials bear the risk of biofilm formation through micro organisms. Most pathogenic micro organisms adhere to cells through the formation of a bond to sugar moieties of glycoproteins present on the surface of the cells. The micro organisms themselves form an envelope composed of polysaccharides, proteins and glycoproteins to which other micro organisms may bind. This leads to the formation of a biofilm. For example, a dextran-film facilitates the binding of streptococcus mutens on dentin (M. T. Madigan, J. M. Martinko, P. V. Dunlap, D. P. Clark, Brock: Biology of the micro organisms, Pearson Benjamin Cummings, 2009, chapter 28, pages 122 following). Since almost all chronic wounds are infected with micro organisms, the use of a polysaccharide in a resorbable wound cover bears the risk of biofilm formation.

A major disadvantage of the known polyurethane foams is that they can not be exactly tailored to the edges of a wound bed. Therefore, they will not completely cover the wound. This has a negative effect on wound healing since fibroblasts or keratinocytes can not adhere to the polyurethane foam on the entire surface of the wound bed.

Another disadvantage of the polyurethane foam described in US 2007/0299151 is that the resorption rate can not be adapted. Advantageously, a wound cover made of polyurethane foam should have a high resorption rate when used on infected and heavily exudating wounds so that medical personal have better control of the condition of the wound every time the wound cover is changed. When used as a cover for wounds showing only a slight production of exudates, the resorption rate of the polyurethane foam should be low, since the wound cover does not need to be changed so frequently. Nevertheless, medical personal should be able to check the condition of the wound every time the wound coverage is changed as to be able to decide if further medical measures have to be taken.

One object of the present invention is therefore to avoid the disadvantages of polyurethane foams known in the state of the art and especially to provide a polyurethane foam wound cover which is biocompatible and resorbable and which specifically shows better covering characteristics on wounds such as to allow an optimal healing process. This objective is achieved with a polyurethane foam cover according to claim 1.

A resorbable polyurethane foam wound cover according to the present invention having open pores, an adaptable resorption rate and being readily adaptable to a wound bed by swelling or by gelling is produced or is producible from a composition comprising:

at least one polyol component comprising at least one compound with at least 2, but preferable 2, 3 or 4 hydroxyl groups;
at least one polyisocyanate component or at least one polyurethane pre-polymer comprising at least one compound with at least 2, but preferably 2, 3 or 4 isocyanate groups;
an additive comprising at least one of the following compounds: polyvinylpyrrolidone, polyvinylpolypyrrolidone, sulfonated copolyester, polyvinylalkylether, a copolymer of polyvinylpyrrolidone with vinylacetate, vinylimidazole or vinylcarpolactame, a copolymer of alkylvinylether with an acid or an anhydrite, preferably maleinic acid or maleinic anhydride, as well as polymers and copolymers of acrylic acid as well as their salts or mixtures thereof.

The composition for the production of the polyurethane foam wound cover according to the present invention may comprise further components. Preferably the composition comprises the following further components:

at least one pore forming agent, preferably comprising a divalent fatty acid salt, especially in the form of a powder;
a pore stabiliser, preferably turkey-red oil or polyethersyloxane;
at least one catalyst;
at least one polycaprolactone, preferably poly(ε-caprolactone);
at least one mono- or polysaccharide, preferably starch.

Preferably a resorbable polyurethane foam according to the present invention should not include any polysaccharides enhancing biofilm formation. Nonetheless, it should comprise a compound which imitates the chemical characteristics of a polysaccharide and which can enhance the adhesion of eukaryotic cells to the foam.

The resorption rate of the polyurethane foam wound cover may be adapted by the following:

by choice of an appropriate polyethylene glycol;
by variation of the ratio of different polyethylene glycols with different average molar masses in a mixture of polyethylene glycols;
by variation of the amount of poly(ε-caprolactone);
by variation of the ratio between the amount of poly(ε-caprolactone) to the amount of the at least one polyethylene glycol, additive c. and water.

The pressure strength of the polyurethane foam may be adapted similarly.

By addition of additive c. and by appropriate choice of the polyol component, the polyurethane foam wound cover will gel or expand upon contact with wound exudate. By this, the foam adapts very well to the edges of a wound bed and can completely fill the wound or wound cavity. The newly formed and ingrowing fibroblasts, ceratinocytes and epithelial cells do not have to bridge any gap between the cover and the edges of the wound bed but instead can directly use the polyurethane foam as artificial extracellular matrix.

Another advantage is that the swelling of the foam leads to a better shock absorbance inside the wound by the polyurethane foam.

Formation of a gel is understood herein as a special form of swelling wherein the foam itself does not dissolve. In this process, the polyol as well as additive c. take up moisture from the wound exudate and bind it.

Additive c., especially polyvinylpyrrolidone, is added in a relatively small concentration to the polyethylene glycol wherein, after polymerisation of the foam, it will be mostly distributed within and on the surface of the foam in small “islands”. This leads to areas where the concentration of additive c., especially polyvinylpyrrolidone, is partially increased. Thus, even if only small concentrations of additive c., especially polyvinylpyrrolidone, are used in the composition, areas with a high enough concentration to lead to a biological activity will be present.

A particular advantage of the use of polyvinylpyrrolidone as additive c. is that it can bind proteins. By this binding the diffusion of growth factors will be hindered and free matrix metalloproteases (NMPs) will be bound, so that the concentration of these molecules will be lowered in the area of the growing cells. This leads to a positive influence on wound healing.

Surprisingly, it has been found that by using polyvinylpyrrolidone in the polyurethane foam, the adhesion of bacteria and biofilm formation can be reduced while adhesion of eukaryotic cells is not hindered. Without restricting the invention on any theory, it is believed that for a successful wound healing an intermediate extra cellular matrix of proteins has to be formed first which serves as an adhesion matrix for the cells. Formation of this intermediate extra cellular matrix is probably positively influenced by the polyvinylpyrrolidone.

Further advantages associated to the use of polyvinylpyrrolidone as replacement for polysaccharides in resorbable polyurethane foams are its good biological compatibility as well as the lack of toxicity.

The polyol component comprises at least one compound with at least two or more hydroxyl groups. Preferably, the polyol component comprises a mixture of two or more compounds with at least two or more hydroxyl groups. Preferred compounds are hydroxyl terminated poly ethers like α,ω-dihydroxy poly(oxyethylene), α,ω-dihydroxy poly(1,2-ethyleneoxide), α,ω-dihydroxy poly(1,2-propyleneoxide), α,ω-dihydroxy poly(1,3-trimethyleneoxide), α,ω-dihydroxy poly(1,4-tetramethyleneoxide), α,ω-dihydroxy poly(methyleneoxy-1,2-ethyleneoxide) and the like as well as copolymers thereof, preferably having molar masses of up to 15′000 g/mol; hydroxyterminated aliphatic polycarbonates like α,ω-dihydroxy poly(ethylenecarbonate), α,ω-dihydroxy poly(1,2-propylenecarbonate), α,ω-dihydroxypoly(1,3-propylenecarbonate), α,ω-dihydroxy poly(tetramethylenecarbonate), α,ω-dihydroxy poly(hexamethylenecarbonate) and the like as well as co-polymers thereof, preferably having molar masses of up to 15′000 g/mol; poly anhydrides from dicarbonic acids, like malonic acid, succinic acid, glutaric acid and the like as well as co-polymer thereof, preferably with molar masses up to 15000 g/mol; low molecular two- or polyvalent alcohols like glycol, polyethylene glycol, 1,2-propylene glycol, 1,3-propylene glycol, butane diol, pentane diol, hexane diol and long-chain linear or branched aliphatic diols, glycerol, triethanol amine, pentaerythritol, 2,2-bis(hydroxymethyl)propanol and the like; amino acid dimers, trimers and oligomers containing hydroxyl groups, e.g. from tyrosine and/or serine; as well as sugar alcohols like sorbitol and other natural compounds or derivatives of natural compounds having at least two hydroxyl groups. Further polyestertriols like castor oil and sulphonated castor oil may be used.

Preferably polyestertriols are added to the polyol component. This will lead to a higher polymerisation density within the polyurethane. Most preferably sulphonated castor oil is added.

Preferably, polyesters with hydroxyl groups are comprised in the polyol component. Examples of such compounds are poly carplolactonediol and poly caprolactonetriol (e.g. available under the trade name Capa from Solvay). Further examples are α,ω-dihydroxy poly(D,L-lactide), α,ω-dihydroxy poly(D-lactide), α,ω-dihydroxy poly(L-lactide), α,ω-dihydroxy poly(glycolide), α,ω-dihydroxy poly (hydroxybutyrate) and other aliphatic polyester and their co-polymers including segmented block co-polymers of polyether and polyester segments, like they are obtainable from reacting high molecular polyesters with hydroxyl terminated poly(alkyleneglycols), as well as mixtures of such polyols.

Most preferably, compounds are used which are biocompatible and which may be resorbed by the body. Examples of such compounds are poly(ε-caprolactone) (PCL), poly(ε-caprolactone-coglycolide-co-DL-lactide), branched and unbranched poly ethylene glycole (PEG), PCL-b-PEG-b-PCL, (α,ω-dihydroxy-oligo(((R)-3-hydroxybutyrate-Co-(R)-3-hydroxyvalerate)-block-ethylene glycol).

In an especially preferred embodiment of the present invention the polyol component comprises a mixture of at least one poly ethylene glycol and an additional poly (ε-caprolactone). The at least one poly ethylene glycol can be branched or unbranched and preferably has an average molar mass of between 100 and 15′000, more preferably of between 300 and 5′000. Preferably, the polyol component comprises a mixture of poly ethylene glycol with different average molar masses. The at least one poly (ε-caprolactone) may be branched or unbranched and preferably has an average molar mass of between 100 and 15′000, most preferably of between 100 and 1′000.

For the production of the polyurethane foam polyurethane pre-polymers may be used. In general the pre-polymers used for the production of a polyurethane foam according to the present invention preferably have a molar mass of between 400 and 15′000, more preferably of between 400 and 10′000, and most preferably between 400 and 1′000.

As an alternative to the polyurethane pre-polymers a polyisocyanate component with at least one compound with at least two isocyanate groups may be used in the composition. The polyisocyanate component preferably comprises at least one biocompatible aliphatic polyisocyanate or at least one compound derived from a biocompatible polyamine. Preferred compounds are: a substituted or unsubstituted alkylenediisocyanate with 3 to 12 carbon atoms like hexamethylenediisocyanate or lysinediisocyanate; substituted or unsubstituted cycloalkylenediisocyanates with 5 to 15 carbon atoms like cyclohexylenediisocyanate; substituted or unsubstituted alkylcycloalkylenediisocyanate with 6 to 18 carbon atoms like isophoronediisocyanate; substituted or unsubstituted aromatic diisocyanates like p-phenylenediisocyanante, toluoyldiisocyanate (all isomers and mixtures thereof), 4,4′-diphenylmethanediisocyanate; as well as isomers, trimers, higher oligomers, uretdions, cyanurates and isocyanurates of these diisocyanates and the like.

Most preferably hexamethylenediisocyanat, 1,6-diisocyanatohexane (HDI), 1,4-diisocyanatobutane (BDI), isophoronediisocyanate (IPDI), dicyclohexylmethanediisocyanate (H12MDI), lysinmethylesterdiisocyanate (LDI) or 4,4′-diphenylmethane-diisocyanate (MDI) is used.

Suitable pore forming agents are non-toxic substances, which are also non-toxic in higher concentrations which will occur when the foam is resorbed. These pore forming agents preferably comprise a divalent metal salt of a long chain fatty acid with 1 to 22 carbon atoms. Preferably, pore forming agents comprising calcium stearate are used. The preferred concentration of the pore forming agent in the composition is in the range of 0.01 to 5 weight percent.

The composition can further comprise foam stabilisers which are preferably non-toxic and show no increased toxicity due to their increased concentration when resorbed by the body. Preferably such stabilisers comprise non-ionic and anionic tensides. For example, a polyethersiloxane, a salt of a fatty acid or a sulphonated fatty acid may be used as foam stabiliser. Most preferably, a salt of a sulphonated fatty acid, most preferably sulphonated castor oil is used as foam stabiliser. The concentration of the foam stabiliser is preferably in the range of between 0.001 to 3 weight percent.

Moreover, the composition may also comprise catalysts which are non-toxic and which show no increased toxicity when resorbed by the body. The catalysts should be able to catalyze the reaction between hydroxyl and isocyanate groups. In a composition of the present invention an organometallic catalyst and/or a tertiary amine catalyst is preferably used. Most preferably, a bismuth catalyst and/or 2′2′-dimorpholinyldiethylether is used. The concentration of the catalysts preferably is in the range of 0.01-1.00 weight percent.

Further components like emulsifiers and the like may also be comprised in the composition.

Additive c. preferably comprises polyvinylpyrrolidone. The polyvinylpyrrolidone can comprise polyvinylpyrrolidone with a defined average molar mass or alternatively may comprise a mixture of polyvinylpyrrolidones with different average molar masses. Preferably polyvinylpyrrolidone with an average molar mass of between 7′000 to 1′000′000 or a mixture thereof is used as dispersion. Most preferably, polyvinylpyrrolidone with an average molar mass of between 40′000 and 60′000 is used (e.g. available under the trade name Luvitec K30 from BASF).

Additive c. alternatively may also comprise co-polymers of vinylpyrrolidone with vinylacetate, vinylimidazole or vinylcaprolactam (e.g. available under the trade name Luvitec VA64W, VA64, VPI55K72W and VPC55K65W from BASF). Most preferably a co-polymer of vinylpyrrolidone and vinylimidazol is used, since the imidazol structure enhances the adhesion to proteins.

Additive c. further may also comprise sulphonated copolyesters (e.g. available from Eastman under the trade name Eastman AQ-polymers), polyvinylalkylether, preferably polyvinylmethylether (available under the trade name Lutonal M40 100% from BASF Company), or polyvinylpolypyrrolidone.

Further compounds which may be comprised in additive c. are co-polymers of alkylvinylether, preferably methyl- or ethylvinylether, with an acid or an anhydrite, preferably maleinic acid or maleinic anhydrite (e.g. available from ISP under the trade name Gantrez AN 169 or Gantrez S). Further, additive c. may comprise co-polymers of polyacrylate and sodium-polyacrylate (as available under the trade name Superabsorber T 5066 F from Degusa).

Preferably, the composition with which the polyurethane foam according to the present invention is produced or is producible comprises between 1 and 96 weight percent, preferably between 30 and 60 weight percent and even more preferably between 35 and 50 weight percent of the at least one polyol component.

Preferably the composition further comprises between 4 and 60 weight percent, more preferably between 40 and 60 weight percent, and most preferably between 45 and 55 weight percent of the at least one isocyanate compound or the polyurethane pre-polymer.

Additionally, the composition further comprises at least one polyvinylpyrrolidone in an amount of between 0.001 to 10.0 weight percent, more preferably between 1.0 and 4.0 weight percent. By varying the ratio of the concentration of polyvinylpyrrolidone, polycaprolactone, poly ethylene glycol and/or additive c., the resorption rate of the polyurethane foam can be adapted. Preferably, the resorption rate may also be adapted by varying the amount of water in the composition.

The polyurethane foam according to the present invention may be used as artificial extra cellular matrix for ingrowing cells on skin or connective tissue wounds and therefore preferably comprises pores. The pores can be connected with each other (open pore structure) or the pores can be isolated (closed pore structure). Preferably, the foam has an open pore structure. The amount of open pores within the foam thereby is at least 70%, but preferably at least 90%. The dry foam has pores with an average largest diameter of between 50 to 600 μm. This allows for the ingrowth of skin and connective tissue cells, fibroblasts, ceratinocytes and epithelial cells. It also provides enough space for the formation of new blood vessels (angiogenesis). Since the polyurethane foam of the present invention forms a gel or swells on contact with fluids, especially with wound exudate, the pores of a wet foam, meaning a foam which is completely moistened by fluid or wound exudate, will have average largest diameters of between 100 and 400 μm. Preferably, the largest diameter of the pores in a wet foam is in between 100 and 250 μm.

The polyurethane foam according to the present invention is preferably used as resorbable wound cover or wound filler material. By the gelling and/or expanding characteristics of the foam, the wound cover perfectly fit in a wound bed. This feature is especially useful if the polyurethane foam is used as a wound filler material, since the foam can then act as an artificial extra cellular matrix whereby newly formed cells may directly adhear to the foam without having to bridge a gap between the edges of the wound bed and the foam first. Such a use is not possible with the polyurethane foams as known in the art, since they are not resorbable and therefore have to be completely removed from the wound or the wound cavity.

The polyvinylpyrrolidone comprised in the foam enhances the adhesion and ingrowth of cells and it also enhances the shock absorbtion characteristic of the foam. Additionally, growth factors as well as matrix metalloproteases (MMPs) are bound by the polyvinylpyrrolidone such that their concentration in the area of the growing cells is reduced. This effect is mostly due to the hygroscopic characteristic of the polyvyinlpyrrolidone molecules.

Biologically active substances or a drug may be added to the polyurethane foam of the present invention by soaking the polyurethane foam with said biologically active substance or said drug and subsequently drying the foam. Through this, the biologically active substance or the drug will crystallize within the pores.

Additionally the surface of the polyurethane foam of the present invention may be modified such as to add a biologically active substance. One possible method is the solid phase synthesis according to Merrifield. Further methods known in the art may also be used to modify the polyurethane foam.

Biologically active substances may be drugs, molecules or compounds which have a biological or chemical effect in the wound. Biologically active substances may be synthetic molecules, biomolecules or mixtures thereof or may comprise enzymes, organic catalysts, ribozymes, organometals, proteins, glycoproteins, peptides, poly aminoacids, antibodies, nucleic acids, steroids, antibiotics, antiviral and antifungal substances, analgesics, immune suppressive substances, cytokines, carbohydrates, oleophobic substances, lipids, components of the extra cellular matrix, drugs or therapeutic agents. Preferably compounds are used which enhance wound healing such as antiseptics, antibiotics, analgetics, polysaccharides, components of the extracellular matrix and growth factors. Suitable growth factors are TGF-β (transforming growth factor β), IGF-I and -II (insulinlike growth factors 1 and 2), PDGF (plateled derived growth factor), EGF (epidermal growth factor), VEGF (vascular endothelial growth factor), KGF (keratinocyte growth factor), FGF-2 (fibroblast growth factor 2), HGF(hepatocyte growth factor) and others. Most preferably a mixture of the growth factors PDGF, VEGF and FGF-2 is used, since these factors enhance angiogenesis.

Additionally, the polyurethane foam of the present invention may comprise a saccharide component, such as a monosaccharide like dextrose, mannose, mannitol, dulcitol, glucose, fructose, galactose and the like; a disaccharide like maltose, lactose, saccharose, cellobiose and the like; an oligo or polysaccharide like cellulose, carboxymethylcellulose, nanocellulose, reduced oxidized cellulose, pektine, amylopectin, amylose, chitin, chitosan, alginate and the like as well as mixtures thereof. Most preferably, starch is comprised in the polyurethane foam of the present invention, since it is cheap and readily available.

In a specially preferred embodiment of the present invention the resorbable polyurethane foam is used as artificial extra cellular matrix for skin and connective tissue lesions. The foam may be used in the form of a roll, a plate or in any other suitable form. In a further specially preferred embodiment, the polyurethane foam may be cut e.g. by medical personal to match the size of the wound.

In an alternative embodiment of the present invention the resorbable polyurethane foam may be used as filling material for a wound cavity. For this use the polyurethane foam is preferably provided in a form of a spiral which can be inserted into the wound cavity as one piece. In the wound cavity the polyurethane foam also serves the function of an artificial extra cellular matrix.

In an alternative embodiment of the present invention the polyurethane foam may also be used as scaffold for cells to be transplanted. In a specially preferred embodiment, skin and connective tissue cells like fibroblasts, cerotynocites and epithetial cells as well as mixed cultures of different cells are used. Stem cells e.g. from skin or from connective tissues may also be used.

A further alternative embodiment provides for a size measurement table on the packaging of the polyurethane foam which facilitates cutting the foam to the right size and shape taking into consideration the swelling characteristics of the foam.

In an additional embodiment the polyurethane foam is applied on a backing film, preferably a polymer film onto which a cutting pattern is printed which facilitates cutting the foam into the right size and shape taking into consideration the swelling characteristics of the foam.

In a special alternative embodiment, the polyurethane foam can be used for the local application of an active substance. Since the foam will be resorbed the active substance will be continuously released to the surrounding area until the foam has entirely disappeared. The polyurethane foam of the present invention is therefore especially suited for the continuous application of an active substance to a wound.

The polyurethane foam of the present invention is preferably produced by a continuous material spray method. In this method the composition is applied under pressure through a spray head onto a support. The components of the composition are directly mixed in the spray head or mixed shortly before injection into the spray head in a mixing chamber. When the composition leaves the spray head a sudden pressure change occurs and the composition spray jet will be torn apart thus forming a spray cone. To expand the foam a pore forming agent or CO₂ produced by the reaction of the compounds of the composition may be used. In a specially preferred embodiment the polyurethane foam is produced in a material spray method where water is used as foaming agent to obtain the porous structure of the foam. The CO₂ which is produced by reacting components leads to pore formation. The composition may be applied onto a conveyer belt or may be foamed into a cast. The polyurethane pieces may then subsequently be cut into pieces with appropriate thickness, preferably of in between 1 and 100 mm, more preferably of in between 2 and 20 mm. Depending on the amount of water present in the composition, the size and quantity of pores may be influenced.

In another alternative embodiment of the present invention a kit of different polyurethane foam wound covers with different absorption rates is provided. The absorption rates of each wound cover may be adapted by varying the amount of carpolactone and/or polyvinylpyrrolidone used in the composition. Preferably the resorption rate may also be varied by the amount of water used as well as by varying the polyole component. A kit with different polyurethane foam covers with different resorption rates allows choosing a wound cover with an optimal absorption rate when treating a wound.

Further advantages and characteristics of the present invention are described in the following description of examples and figures.

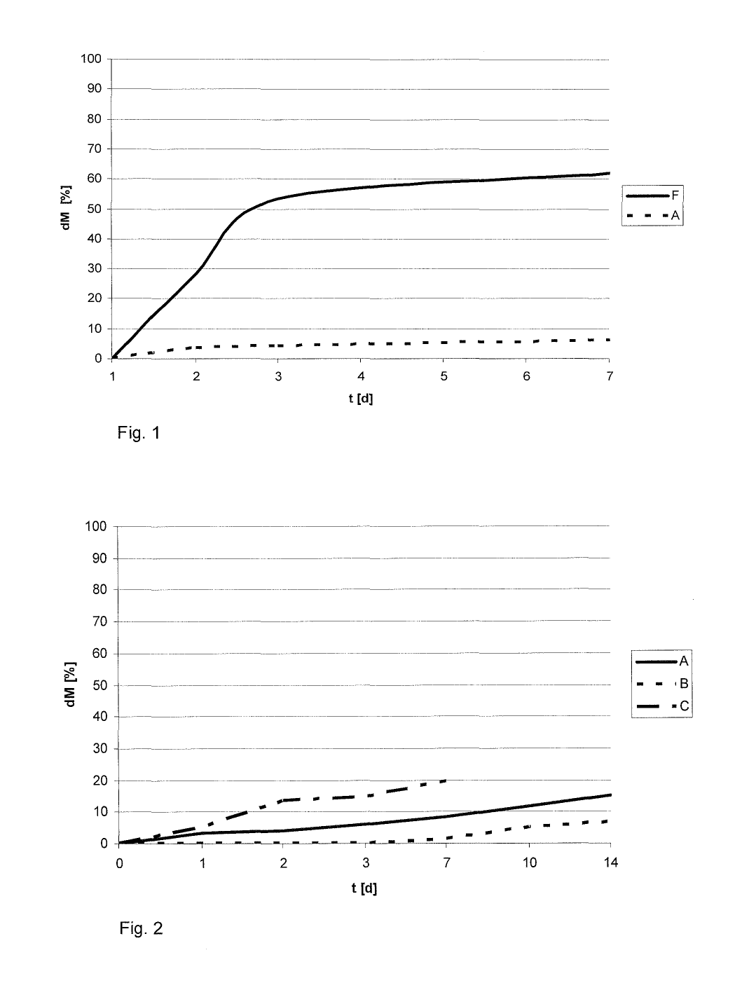

FIG. 1 comparison of the resorption rates of a foam with 2% PVP with the resorption rate of a foam without PVP,

FIG. 2 comparison of resorption rates of foams with different ratios of PEG and polycaprolactone,

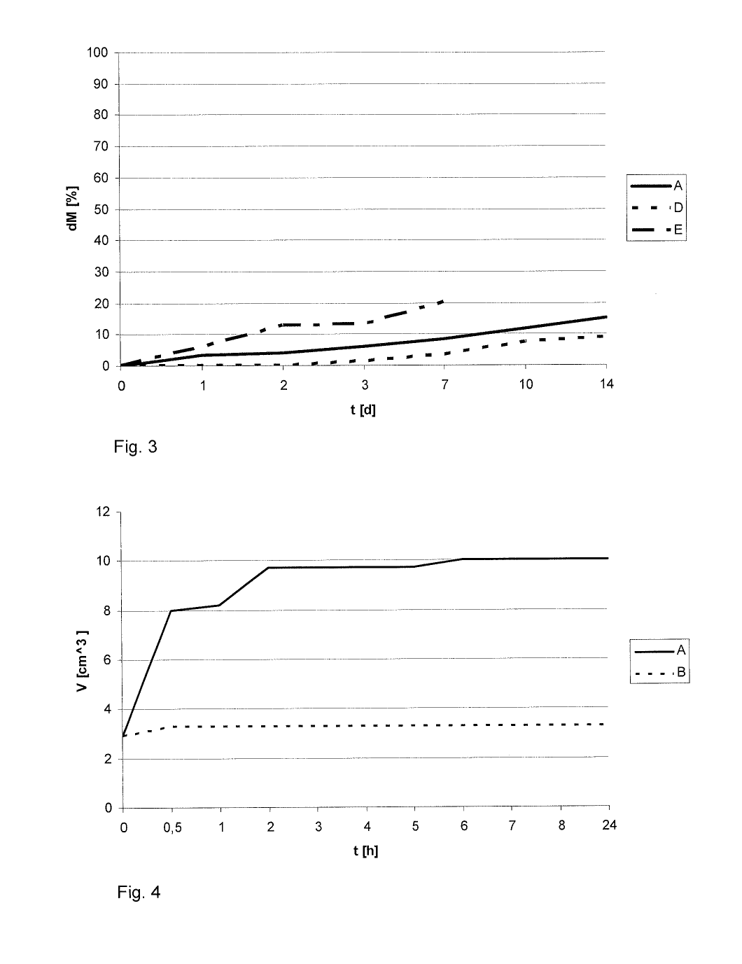

FIG. 3 influence of the substitution of unbranched with branched polycaprolactone under variation of the ratio of polycaprolactone/PEG,

FIG. 4 swelling characteristics of foams with different ratios of polycaprolactone/PEG,

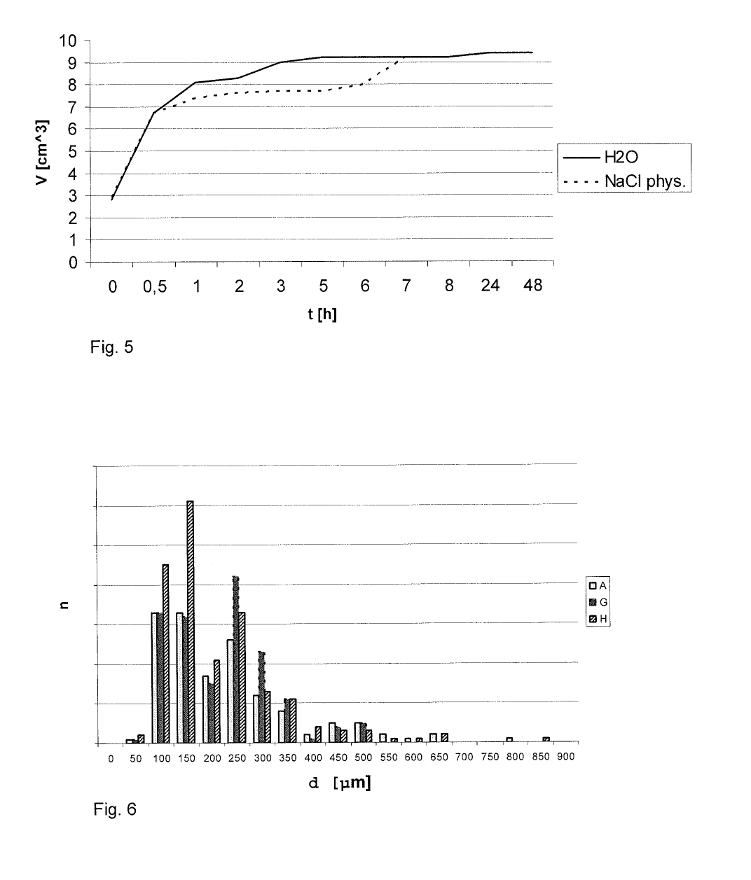

FIG. 5 comparison of the swelling characteristics of a polyurethane foam of the present invention in water and in physiological solution,

FIG. 6 influence of the amount of water in the composition on the size and quantity of pores,

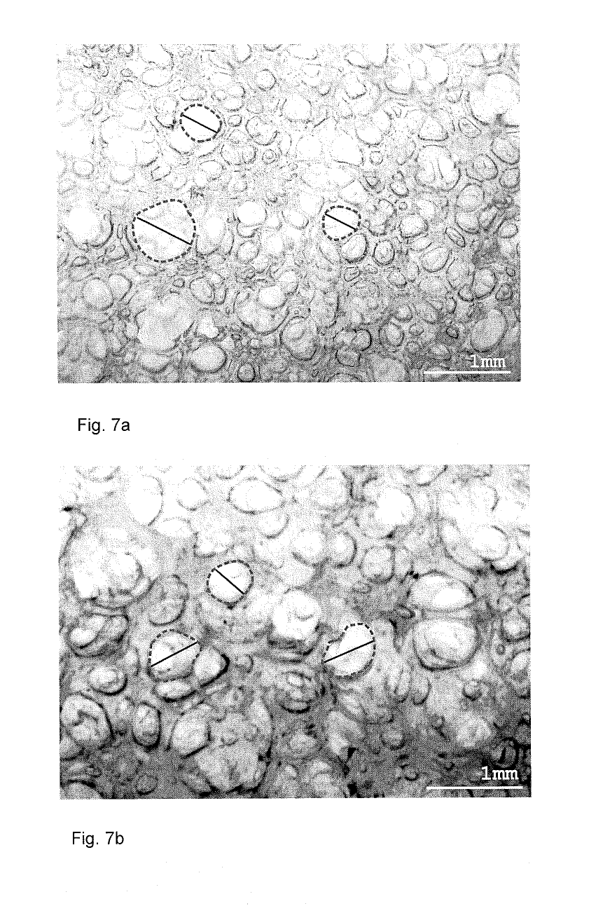

FIG. 7 comparison between the pore structure of a dry and a wet foam, and

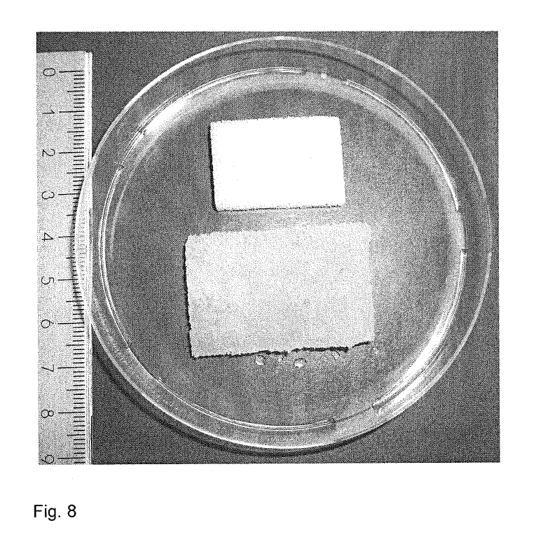

FIG. 8 size increase of a foam of the present invention after two days in water at room temperature.

In the following, resorbtion rates and swelling characteristics of polyurethane foams with different concentrations of PEG, polycaprolactone and polyvinylpyrrolidone were measured. An overview of the different foam compositions is given in the table below (all indications in gram):

| | Composition | | | | | | | |
|---|---|---|---|---|---|---|---|---|
| Component | A | B | C | D | E | F | G | H |
| PEG-400 | 8 | 8 | 8 | 8 | 8 | 8 | 8 | 8 |
| PEG-4000 | 12 | 0 | 6 | 0 | 6 | 12 | 12 | 12 |
| Sympatens | 0.02 | 0.02 | 0.02 | 0.02 | 0.02 | 0.02 | 0.02 | 0.02 |
| TRH/400 | | | | | | | | |
| CAPA 2402 | 0 | 12 | 6 | 0 | | 0 | 0 | 0 |
| CAPA 4801 | 0 | 0 | 0 | 12 | 6 | 0 | 0 | 0 |
| Sulphonated | 4 | 4 | 4 | 4 | 4 | 4 | 4 | 4 |
| castor oil | | | | | | | | |
| Coscat 83 | 0.28 | 0.28 | 0.28 | 0.28 | 0.28 | 0.28 | 0.28 | 0.28 |
| Jeffcat | 0.23 | 0.23 | 0.23 | 0.23 | 0.23 | 0.23 | 0.23 | 0.23 |
| DMDEE | | | | | | | | |
| PVP | 1.06 | 1.06 | 1.06 | 1.06 | 1.06 | 0 | 1.06 | 1.06 |
| Ca-Stearate | 0.5 | 0.5 | 0.5 | 0.5 | 0.5 | 0.5 | 0.5 | 0.5 |
| turkey-red | 0.1 | 0 | 0 | 0 | 0 | 0.1 | 0.2 | 0.2 |
| oil | | | | | | | | |
| Water | 0.3 | 0.3 | 0.3 | 0.3 | 0.3 | 0.3 | 0.25 | 0.2 |
| Desmodur E | 26.5 | 26.5 | 26.5 | 26.5 | 26.5 | 26.5 | 24.75 | 23.0 |
| 305 | | | | | | | | |

Production of foams used in the following examples always followed the same protocol. The open pores were formed by the use of water as foaming agent (formation of CO₂) with calcium stearate as pore forming agent. Polyvinylpryrrolidone, if present in the composition, was first dissolved in PEG-400 at 170° C. After the dissolution of PVP, the other components like sulphonated castor oil (poly estertriol), Sympatens TRH/400 (Kolb AG, Hedingen, Germany) as emulsifier and PEG and/or PVP or mixtures thereof were added and dissolved at 170° C. The mixture was subsequently cooled to 70° C. in a circulation air oven. In a next step, a mixture containing water, the catalysts Coscat 83 (Vertellus Inc., Indianapolis, USA) and Jeffcat (Huntsman Performance Products, The Woodlands, Tex., USA), calcium stearate as pore forming agent as well as turkey-red oil as additional emulsifier was added. Desmodur E 305 (Bayer Material Science AG, Leverkusen, Germany) pre-heated at 70° C. was added to the mixture followed by intensive mixing for 1 minute (in a PP 200 beaker with a SpeedMixer™ mixer). The beaker was subsequently covered (a small hole for pressure equalization was provided) and put in a heating chamber at 110° C. for 15 minutes followed by 3 hours at 70° C. After cooling to room temperature, the foam body was removed and cut into pieces having identical dimensions.

To determine the resorption rate the loss of mass of the foam was calculated. This was done according to a protocol on oxidative degradation of polymers in medicinal products according to ISO Norm 10993-13:1999. A solution of 3% hydrogen peroxide was used as liquid media. The samples were dried under vacuum until they showed a constant weight and subsequently put in a Petri dish with a 3% solution of hydrogen peroxide followed by incubation at 37° C. During the test, the samples were constantly shaken on a lab shaker. At different time points the samples were removed and dried under vacuum until they showed a stable weight. From the measured weight the loss of mass could be calculated. As positive control the resorbable wound cover “Promogran Prisma” (Johnson & Johnson) which, according to the manufacturer, has an in vivo resorption rate of between 2 to 3 days, and wound sutures “Monocryl ungefärbt” (Johnson & Johnson) which have an in vivo resorption rate of between 90 and 120 days according to the manufacturer, were used. The non resorbable foam “Corpura MCF 0.3 (Corpura)” was used as a negative control.

FIG. 1 shows a diagram with the resorption rate of a foam with 2 weight percent PVP (composition A) as well as the absorption rate a foam without PVP (composition F). The loss of mass dM of the foam is given in percent of the initial mass and is shown in relation to time t in days. It may be seen that the resorption rate of a polyurethane foam without PVP is higher than the resorption rate of a foam with PVP. The PVP therefore does not only serve for the gelling of the foam but can also be used to influence the resorption rate.

FIG. 2 shows a diagram with a resorption rate of a foam with 22.7 weight percent (12 g) PEG-4000 (Kolb AG, Hedingen, Germany) according to composition A, a foam with 22.7 weight percent unbranched polycaprolactone with an average molar mass of 4000 (CAPA 2402, Perstrop UK Ltd, Warrington, UK) according to composition B as well as of a foam with both 11.35 weight percent PEG-4000 and CAPA 2402 according to composition C. All compositions comprise 15 weight percent (8 g) PEG-400 (Kolb AG, Hedingen, Germany). Here it may be seen that the resorption rate of a polyurethane foam according to the present invention can be adapted by choosing the right ratio between PEG and polycaprolactone. Further, the figure shows that polyurethane foams with a mixture of PEG with polycaprolactone (composition C) have a higher resorption rate that foams with only PEG (composition A) or only with polycaprolactone (composition B).

FIG. 3 shows a diagram with the resorption rate of a polyurethane foam with 22.7 weight percent (12 g) PEG-4000 (Kolb AG, Hedingen, Germany) according to composition A, a polyurethane foam with 22.7 weight percent tetrafunctional polycaprolactone with an average molar mass of 8000 (CAPA 4801 of Perstrop UK Ltd., Warrington, UK) according to composition D as well as of a foam with 11.35 weight percent PEG-4000 and CAPA 4801 according to composition E. All compositions comprise 15 weight percent (8 g) PEG-400 (Kolb AG, Hedingen, Germany). It may be seen that the resorption rate of a polyurethane foam according to the present invention can be influenced by varying the ratio of PEG and polycaprolactone.

FIG. 4 shows a diagram showing the different swelling characteristics of two different polyurethane foams of the present invention. Exemplarily the swelling characteristics of foams with the compositions A and B were measured. The foam samples were first dried under vacuum until their weight was constant and subsequently put into a physiological solution for 24 hours. The volume V was measured in cm³. As can be seen on the figure, substitution of PEG-4000 with a linear polycaprolactone CAPA2402 has the result that the foam only swells to a smaller volume. Therefore it is not only possible to vary the resorption rate of the foam but also to vary the swelling characteristics thereof by varying the mixing ratio between PEG and polycaprolactone.

FIG. 5 shows the swelling characteristics of polyurethane foams according to the present invention in water and physiological solution, here exemplarily with a foam produced with composition G. It can be seen that the increase of the volume V (in cm³) depends mainly on the solution (water or physiological solution) used.

FIG. 6 shows the influence of the amount of water in the composition on the size and quantity of pores in the foam. The analysis was made with dry and wet foam samples produced with compositions A, G and H. A stereo microscope Wild M3Z (Wild Leitz) with a cold light source Intralux 4000 (Wild Leitz) and a digital camera was used. The diameter was measured using a ruler. The largest diameter d of the pores was measured in an area of 20.5 mm² of the samples. The diagram shows the distribution of the largest diameter of pores in the wet foams. The absolute number n of the pores having a largest diameter is shown in size intervals of 50 μm. It can be seen that pore sizes may be varied by variation of the amount of water in the composition. When using a small amount of water like in compositions G and H the amount of pores which have a largest diameter in the target area of in between 200 and 250 μm in the wet foam is increased. Since the polyurethane foam is used as wound cover it will be exposed to wound exudate. Therefore the pore diameter of the wet foam is decisive for an improved wound healing. The pores should have an average largest diameter of in between 50 and 450 μm preferably of in between 200 and 250 μm, so that skin and connective tissue cells can optimally grow into the foam.

FIG. 7 shows the differences of the pore structure of a dry foam (FIG. 7a) and of a wet foam (FIG. 7b). The foam was produced with composition G. On this figure, three pores have exemplarily been marked with a dashed line. Further, for each of these pores, the largest diameter is marked with a line.

FIG. 8 shows two pieces of a polyurethane foam according to the present invention which were produced with the same composition. The piece on top of the picture is dry while the piece on the bottom is wet. The initial size of both foam pieces was identical. It can be clearly seen that the polyurethane foams according to the present invention swell when they are contacted with fluids.

1-15. (canceled)

16. A polyurethane foam wound cover produced or producible from a composition comprising
a. at least one polyol component comprising at least one compound with at least two hydroxyl groups;
b. at least one polyisocyanate component or at least one polyurethaneprepolymer comprising at least one compound with at least two isocyanate groups; and
c. an additive comprising at least one of the following compounds: polyvinylpyrrolidone, polyvinylpolypyrrolidone, sulfonated copolyester, polyvinylalkylether, a copolymer of polyvinylpyrrolidone with vinylacetate, vinylimidazole or vinylcaprolactam, a copolymer of alkylvinylether with an acid or an anhydrite as well as polymers and copolymers of acrylic acid and their salts or mixtures thereof.

17. A polyurethane foam wound cover according to claim 16, wherein the composition further comprises at least one of the following components:
d. at least one pore forming agent;
e. a pore stabilizer;
f. at least one catalyst;
g. at least one polycaprolactone;
h. at least one mono- or polysaccharide.

18. A polyurethane foam wound cover according to claim 16, wherein the at least one polyol component is present in an amount between 1% and 96% by weight.

19. A polyurethane foam wound cover according to claim 16, wherein the at least one polyisocyanate component or the at least one polyurethane prepolymer are present in an amount of between 4% and 60% by weight.

20. A polyurethane foam wound cover according to claim 16, wherein the additive c. is present in an amount of between 0.001% and 10% by weight.

21. A polyurethane foam wound cover according to claim 16, wherein the polyvinylpyrrolidone has a molecular weight of between 7,000 and 1,000,000 g/mol.

22. A polyurethane foam wound cover according to claim 16, wherein the at least one polyol component comprises at least one polyethyleneglycol.

23. A polyurethane foam wound cover according to claim 16, wherein the at least one polyol component comprises at least one polycaprolactone.

24. A polyurethane foam wound cover according to claim 16, wherein the foam essentially comprises open pores.

25. A polyurethane foam wound cover according to claim 16, wherein the foam comprises a biologically active compound.

26. A polyurethane foam wound cover according to claim 16, wherein a pattern is provided on the foam which enables the cutting of the foam to specified dimensions and forms taking into consideration the swelling properties of the foam.

27. A method of production of a polyurethane foam wound cover according to claim 16, wherein the foam is produced by a material spray method followed by expansion of the foam.

28. A method of production of a polyurethane foam wound cover according to claim 16, wherein the foam is produced by a continuous material spray method.

29. A method of production of a polyurethane foam wound cover according to claim 16, wherein the foam is foamed in a mould.

30. A kit with polyurethane foam wound covers with different resorption rates, wherein the resorption rates of the polyurethane foam wound covers is adjusted by the amount of polycarpolactone and/or additive c used in the composition.

31. A polyurethane foam wound cover according to claim 16, wherein the additive c is maleinic acid or maleinic anhydrite.

32. A polyurethane foam wound cover according to claim 24, wherein the pores have a diameter of between 50 and 400 μm in wet condition.

33. A polyurethane foam wound cover according to claim 25, wherein the biologically active compound is provided in crystalline form.
